(19) United States
(12) Patent Application Publication
(10) Pub. No.: US 2021/0361638 A1
(43) Pub. Date: Nov. 25, 2021

(54) FXR AGONISTS FOR THE TREATMENT OF LIVER DISEASES

(71) Applicant: NOVARTIS AG, Basel (CH)

(72) Inventors: Michael Badman, Newton, MA (US); Clifford Brass, New York, NY (US); Bryan Laffitte, Cardiff, CA (US)

(21) Appl. No.: 16/767,078

(22) PCT Filed: Nov. 28, 2018
(86) PCT No.: PCT/IB2018/059383

§ 371 Date: May 26, 2020

Related U.S. Application Data
(60) Provisional application No. 62/593,084, filed on Nov. 30, 2017.

(51) Int. Cl. A61K 31/46; A61K 47/54; A61P 1/16

(52) U.S. Cl. CPC A61K 31/46; A61P 1/16; A61K 47/549

(57) ABSTRACT

The invention provides methods for treating or preventing a condition mediated by the farnesoid X receptor (FXR), comprising administering tropifexor, a pharmaceutically acceptable salt, an amino acid conjugate or an acyl glucuronide conjugate thereof, at a dose of about 140 μg to about 250 μg, to a subject in need thereof.

### FIELD OF THE INVENTION

The present invention relates to novel regimens for treating or preventing liver conditions mediated by farnesoid X receptors (FXRs), by using therapeutically effective amount of a FXR agonist, e.g. tropifexor as well as methods, uses, compositions involving such regimens.

### BACKGROUND OF THE INVENTION

FXR agonism has shown clinical benefits in subjects with cholestatic disorders (Nevens et al., J. Hepatol. 60 (1 SUPPL. 1): 347A-348A (2014)), bile acid malabsorption diarrhea (Walters et al., Aliment Pharmacol. Ther. 41(1):54-64 (2014)) and non-alcoholic steatohepatitis (NASH; Neuschwander-Tetri et al 2015).

Obeticholic acid (6α-ethyl-chenodeoxycholic acid), that is abbreviated to OCA and also known as INT-747, is a bile acid-derived FXR agonist, analogue to the natural bile acid chenodeoxycholic acid. In clinical studies, OCA showed efficacy in both Primary Biliary Cirrhosis (PBC) and non-alcoholic steatohepatitis (NASH) subjects; however OCA treatment may be associated with increased pruritus. OCA was tested at doses between 5 mg and 50 mg in PBC subjects or NASH subjects. In the FLINT trial, 35% of OCA-treated patients showed improvement in fibrosis compared with 19% of placebo-treated patients; however, no significant change in NASH resolution was observed compared to placebo. Furthermore, pruritus was more common in OCA-treated patients (23%) compared to placebo-treated patients (6%).

There remains a need for new treatments and therapies for liver conditions mediated by FXR, which are effective and could be associated with more limited side effects.

### SUMMARY OF THE INVENTION

The invention relates to methods of treating, preventing, or ameliorating conditions mediated by farnesoid X receptors (FXR), in particular liver diseases, comprising administering to a subject in need thereof a therapeutically effective amount of a FXR agonist of formula (I)

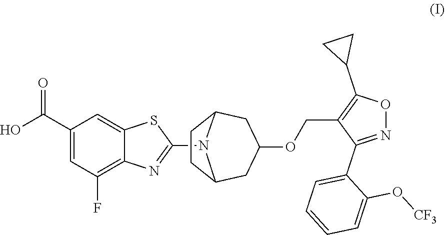 [SMILES: O=C(O)c1cc(F)c2nc(N3C4CCC3CC(OCc3c(-c5ccccc5OC(F)(F)F)noc3C3CC3)C4)sc2c1]

(i.e. 2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid), a stereoisomer, an enantiomer, a pharmaceutically acceptable salt or an amino acid conjugate thereof, e.g. a FXR agonist of formula (II)

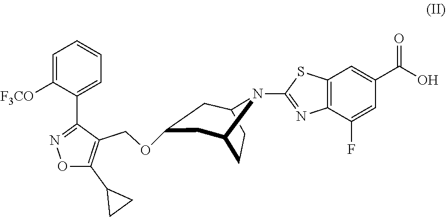

(i.e. 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid) (as herein defined as Compound A, or tropifexor), in free form, or a pharmaceutically acceptable salt or an amino acid conjugate thereof.

The invention further provides new dosing regimens of tropifexor or amino acid conjugate thereof, e.g. glycine conjugate, taurine conjugate or acyl glucuronide conjugate of tropifexor for treating or preventing liver diseases and disorders mediated by farnesoid X receptors (FXR), as well as the use of such new regimens and pharmaceutical compositions adapted for administering such new regimens. Such new dosing regimens are effective and well tolerated regimens for treating or preventing liver diseases and disorders mediated by farnesoid X receptors (FXR) in humans.

In comparison to OCA, the non-bile acid FXR agonists disclosed herein, e.g. tropifexor is ˜300×more potent, with no FGR5 effects therefore has a greater specificity when administered to a patient in need thereof.

The compounds of formula (I) (e.g. tropifexor) are non-bile acid derived FXR agonists. They are described in WO2012/087519.

Non-bile acid derived FXR agonists have the advantages of greater potency, greater specificity for the FXR target and absorption, distribution, metabolism and elimination processes that are not subject to processes of bile acid metabolism.

Various (enumerated) embodiments of the present invention are described herein. It will be recognized that features specified in each embodiment may be combined with other specified features to provide further embodiments of the present disclosure.

Embodiment 1: Therapeutic regimens for treating or preventing a condition mediated by Farnesoid X receptor (FXR), comprising administering the FXR agonist of formula (I), a stereoisomer, an enantiomer, a pharmaceutically acceptable salt thereof or an amino acid conjugate thereof, e.g. tropifexor, e.g in free form or an amino acid conjugate thereof, at a dose (e.g. daily dose) of about 140 μg to about 250 μg, about 140 μg to about 200 μg. Such doses may be for daily or twice daily administration.

Embodiment 2: Therapeutic regimens for treating or preventing a condition mediated by Farnesoid X receptor (FXR), comprising administering tropifexor, e.g in free form or an amino acid conjugate thereof, at a dose of about 140 μg, about 150 μg, about 160 μg, about 170 μg, about 180 μg, about 190 μg, about 200 μg, about 210 μg, about 220 μg, about 230 μg, about 240 μg or about 250 μg. Such doses may be for daily administration (e.g. daily doses). Such doses may be for daily or twice daily.

Embodiment 3: Therapeutic regimens for treating or preventing a condition mediated by Farnesoid X receptor (FXR) such as a liver or an intestinal disease, comprising administering tropifexor or an amino acid conjugate thereof, at a dose of about 140 μg, e.g. daily or twice daily, e.g. for daily administration.

Embodiment 4: Therapeutic regimens for treating or preventing a condition mediated by Farnesoid X receptor (FXR) such as a liver or an intestinal disease, comprising administering tropifexor or an amino acid conjugate thereof, at a dose of about 140 μg or about 200 μg, e.g. daily or twice daily, e.g. for daily administration.

Embodiment 5: Therapeutic regimens for treating or preventing a condition mediated by Farnesoid X receptor (FXR) such as a liver or an intestinal disease, comprising administering tropifexor or an amino acid conjugate thereof, at a daily dose of about 200 μg, e.g. daily or twice daily, e.g. for daily administration.

Embodiment 6: Therapeutic regimens for treating or preventing a condition mediated by Farnesoid X receptor (FXR) such as a liver or an intestinal disease, comprising administering tropifexor or an amino acid conjugate thereof, at a daily dose of about 250 μg, e.g. daily or twice daily, e.g. for daily administration.

Embodiment 7: Use of tropifexor or an amino acid conjugate thereof, in the manufacture of a medicament for treating or preventing a condition mediated by Farnesoid X receptor (FXR), wherein tropifexor is to be administered at a dose (e.g. daily dose), of about 140 μg to about 250 μg, about 140 μg to about 200 μg. Such doses may be for administration daily (daily doses) or twice daily, e.g. for daily administration.

Embodiment 8: Use of tropifexor or an amino acid conjugate thereof, in the manufacture of a medicament for treating or preventing a condition mediated by Farnesoid X receptor (FXR), wherein tropifexor is to be administered at a dose of about 140 μg, about 150 μg, about 160 μg, about 170 μg, about 180 μg, about 190 μg, about 200 μg, about 210 μg, about 220 μg, about 230 μg, about 240 μg or about 250 μg. Such doses may be for daily administration (e.g. daily doses) or daily or twice daily, e.g. for daily administration.

Embodiment 9: Use of tropifexor or an amino acid conjugate thereof, in the manufacture of a medicament for treating or preventing a condition mediated by Farnesoid X receptor (FXR), wherein tropifexor is to be administered at a dose of about 140 μg/day to about 250 μg/day, about 140 μg/day to about 200 μg/day.

Embodiment 10: Use of tropifexor or an amino acid conjugate thereof, in the manufacture of a medicament for treating or preventing a condition mediated by Farnesoid X receptor (FXR), wherein tropifexor is to be administered at a dose of about 140 μg, about 150 μg, about 160 μg, about 170 μg, about 180 μg, about 190 μg, about 200 μg, about 210 μg, about 220 μg, about 230 μg, about 240 μg or about 250 μg. Such doses may be for daily administration (e.g. daily doses) or twice daily administration.

Embodiment 11: Tropifexor, e.g. in free form or an amino acid conjugate thereof, for use in treating or preventing a condition mediated by FXR; wherein tropifexor is to be administered at a dose (e.g. daily dose) of about 140 μg to about 250 μg, about 140 μg to about 200 μg, and wherein said condition mediated by FXR is non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis.

Embodiment 12: Tropifexor, e.g in free form or an amino acid conjugate thereof, for use in treating or preventing a condition mediated by FXR; wherein tropifexor is to be administered at a dose of about 140 μg, about 150 μg, about 160 μg, about 170 μg, about 180 μg, about 190 μg, about 200 μg, about 210 μg, about 220 μg, about 230 μg, about 240 μg or about 250 μg. Such doses may be for daily administration (e.g. daily doses). Such doses may be for twice daily administration.

Embodiment 13: The use of tropifexor or an amino acid conjugate thereof, according to any one of Embodiments 1 to 12, wherein the condition mediated by FXR is non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis.

Embodiment 14: The use of tropifexor or an amino acid conjugate thereof, according to any one of Embodiments 1 to 12, wherein the condition mediated by FXR is NAFLD or NASH.

Embodiment 15: A method for treating or preventing a condition mediated by Farnesoid X receptor (FXR) in a subject suffering therefrom, comprising administering to the subject tropifexor or an amino acid conjugate thereof; wherein tropifexor is to be administered at a daily dose of about 140 μg to about 250 μg, about 140 μg to about 200 μg.

Embodiment 16: A method for treating or preventing a condition mediated by Farnesoid X receptor (FXR) in a subject suffering therefrom, comprising administering to the subject tropifexor or an amino acid conjugate thereof; wherein tropifexor is to be administered at a dose of about 140 μg/day to about 250 μg/day, about 140 μg/day to about 200 μg/day.

Embodiment 17: A method for treating or preventing a condition mediated by Farnesoid X receptor (FXR) in a subject suffering therefrom, comprising administering to the subject tropifexor or an amino acid conjugate thereof; wherein tropifexor is to be administered at a dose of about 140 μg/day, about 150 μg/day, about 160 μg/day, about 170 μg/day, about 180 μg/day, about 190 μg/day, about 200 μg/day, about 210 μg/day, about 220 μg/day, about 230 μg/day, about 240 μg/day or about 250 μg/day.

Embodiment 18: A method for treating or preventing a condition mediated by Farnesoid X receptor (FXR) according to any one of Embodiments 1 to 16, wherein the condition is a chronic liver disease, such as e.g. non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, or liver fibrosis.

Embodiment 19: A method for treating or preventing a chronic liver disease, in a subject suffering therefrom, comprising administering to the subject tropifexor or an amino acid conjugate thereof, in a dose (e.g. daily dose) of about 140 μg to about 250 μg, about 140 μg to about 200 μg.

Embodiment 20: A method for treating or preventing a chronic liver disease in a subject suffering therefrom, comprising administering to the subject tropifexor or an amino acid conjugate thereof, at a dose of about 140 μg, about 150 μg, about 160 μg, about 170 μg, about 180 μg, about 190 μg, about 200 μg, about 210 μg, about 220 μg, about 230 μg, about 240 μg or about 250 μg. Such doses may be for daily administration (e.g. daily doses). Such doses may be for once daily or twice daily administration.

Embodiment 21: A method according to Embodiment 19 or 20 for treating pr preventing a liver disease or disorder selected from non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis and liver fibrosis.

Embodiment 22: A method according to Embodiment 19 or 20 for treating or preventing NASH.

Embodiment 23: A use, tropifexor or a method for treating or preventing a condition mediated by Farnesoid X receptor (FXR), e.g. a chronic liver disease, in a subject suffering therefrom according to any one of Embodiments 1 to 22, wherein tropifexor is to be administered for a period of 3 months to lifelong, e.g. 6 months to lifelong, e.g. 1 year to lifelong, e.g. for a period of 3 months to 1 year, e.g. 6 months to lifelong, e.g. for a period of 3 months, 6 months or 1 year or for lifelong.

Embodiment 24: A use, tropifexor or a method according to any one of Embodiments 1 to 23, for treating or preventing a liver disease or disorder selected from non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis and liver fibrosis.

Embodiment 25: A use, tropifexor or a method according to any one of Embodiments 1 to 23, for treating or preventing non-alcoholic steatohepatitis (NASH), and wherein NASH is mild to moderate with fibrosis level F2-F3.

Embodiment 26: A use, tropifexor or a method according to any one of Embodiments 1 to 23, for treating or preventing non-alcoholic steatohepatitis (NASH), wherein NASH is confirmed based on liver biopsy obtained 2 years or less before treatment intitiation with tropifexor (also called biopsy-proven NASH) and NASH is mild to moderate with fibrosis level F2-F3.

Embodiment 27: A use, tropifexor or a method according to any one of Embodiments 1 to 23, for treating or preventing non-alcoholic steatohepatitis (NASH), wherein presence of NASH has been demonstrated by:

i) by one of the following: Histologic evidence of NASH based on liver biopsy obtained 2 years or less before treatment with a FXR agonist according to any one of Embodiments 1 to 23, with a diagnosis consistent with NASH, fibrosis level F1, F2 or F3, no diagnosis of alternative chronic liver diseases and ALT≥60 IU/L (males) or ≥40 IU/L (females), or

ii) Phenotypic diagnosis of NASH based on presence of all three of the following:
ALT≥60 IU/L (males) or 40 IU/L (females) and
BMI≥27 kg/m2 (in patients with a self-identified race other than Asian) or ≥23 kg/m2 (in patients with a self-identified Asian race) and
Diagnosis of Type 2 diabetes mellitus by having either: HbA1C≥6.5% or Drug therapy for Type 2 diabetes mellitus.

Embodiment 28: A pharmaceutical unit dosage form composition comprising about 140 μg, about 150 μg, about 160 μg, about 170 μg, about 180 μg, about 190 μg, about 200 μg, about 210 μg, about 220 μg, about 230 μg, about 240 μg or about 250 μg of tropifexor suitable for oral administration up to a maximum total dose of 500 μg per day. Such unit dosage form compositions may be in a form selected from a liquid, a tablet, a capsule. Also these unit dosage form compositions are for use in treating a chronic liver disease, e.g. non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, e.g. for use in treating non-alcoholic steatohepatitis (NASH), e.g. for use in treating phenotypic non-alcoholic steatohepatitis (NASH).

Embodiment 29: A use, tropifexor or a method according to any one of Embodiments 1 to 27, a pharmaceutical unit dosage form of Embodiment 28, is administered to humans in a fasting state, e.g. administration in a fasting state, at least 30 minutes prior to first beverage, apart from water, and at least 60 minutes prior to the first meal of the day.

Embodiment 30: A use, tropifexor or a method according to any one of Embodiments 1 to 27, a pharmaceutical unit dosage form of Embodiment 28, is administered to humans with impaired hepatic function and wherein tropifexor or an amino acid conjugate thereof, is administered at reduced dose compared to the dose administered to humans without impaired hepatic function. Such impaired hepatic function may be, for example classified by the Child-Pugh system: mild (Child-Pugh A), moderate (Child-Pugh B), severe (Child-Pugh C).

### DETAILED DESCRIPTION OF THE INVENTION

### Definitions

For purposes of interpreting this specification, the following definitions will apply and whenever appropriate, terms used in the singular will also include the plural and vice versa.

As used herein, the term “about” in relation to a numerical value×means+/−10%, unless the context dictates otherwise.

As used herein, the term “FXR agonist” refers to an agent that directly binds to and upregulates the activity of FXR.

As used herein, the term “pharmaceutically acceptable” means a nontoxic material that does not interfere with the effectiveness of the biological activity of the active ingredient(s).

As used herein, the term “amino acid conjugate” refers to conjugates of the compound of Formula (I) with any suitable amino acid. Preferably, such suitable amino acid conjugates of the compound of Formula (I) will have the added advantage of enhanced integrity in bile or intestinal fluids. Suitable amino acids include but are not limited to glycine, taurine and acylglucuronide. Thus, the present invention encompasses the glycine, taurine and acylglucuronide conjugates of the compound of Formula (I), e.g. glycine, taurine and acylglucuronide conjugates of tropifexor.

As used herein, the term “subject” or “subject” refers to a human.

As used herein, the term “treat”, “treating” or “treatment” in connection to a disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. The term “alleviating” or “alleviation”, for example in reference to a symptom of a condition, as used herein, refers to reducing at least one of the frequency and amplitude of a symptom of a condition in a patient. In one embodiment, the terms “method for the treatment” or “method for treating”, as used herein, refer to “method to treat”.

As used herein, the term “therapeutically effective amount” refers to an amount of the compound of the invention, e.g. compound of formula (I) or a pharmaceutically acceptable salt thereof, e.g. tropifexor, which is sufficient to achieve the stated effect. Accordingly, a therapeutically effective amount of a FXR agonist of formula (I), a stereoisomer, an enantiomer, a pharmaceutically acceptable salt thereof or an amino acid conjugate thereof, e.g. tropifexor or an amino acid conjugate thereof, used for the treatment or prevention of a condition mediated by FXR will be an amount sufficient for the treatment or prevention of the condition mediated by FXR.

By “therapeutic regimen” is meant the pattern of treatment of an illness, e.g., the pattern of dosing used during the treatment of the disease or disorder.

As used herein, a subject is “in need of” a treatment if such subject would benefit biologically, medically or in quality of life from such treatment.

As used herein, the term “liver disease or disorder” encompasses one, a plurality, or all of non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis and liver fibrosis.

As used herein, a NASH phenotype or phenotypic NASH can be described using combinations of several features of metabolic syndrome (obesity, Type 2 diabetes mellitus) along with elevated ALT/AST and fatty infiltration of the liver.

As used herein, fibrosis can be staged using scoring systems described in the literature, for example the most commonly used in the United States are the Knodell histologic activity index (0-4), Batts-Ludwig stage (0-4) and Scheuer (0-4) (3-5) and the METAVIR scheme (0-4) in Europe. The Knodell and METAVIR score fibrosis from stage 0-4, with stage 4 as cirrhosis, whereas Ishak scores fibrosis from 0-6 where 5 is incomplete or early cirrhosis and 6 indicates established cirrhosis.

As used herein, NAS is NAFLD Activity Score, and can be described as a semi-quantitative instrument used to judge treatment response and disease progression in patients. As used herein, a “therapeutically effective amount” refers to an amount of compound of formula (I), a stereoisomer, an enantiomer, a pharmaceutically acceptable salt thereof or an amino acid conjugate thereof, e.g. tropifexor or an amino acid conjugate thereof, e.g. tropifexor, that is effective, upon single or multiple dose administration to a subject (such as a human subject) at treating, preventing, curing, delaying, reducing the severity of, ameliorating at least one symptom of a disorder or recurring disorder, or prolonging the survival of the subject beyond that expected in the absence of such a treatment.

### DESCRIPTION OF THE FIGURES

FIG. 1. shows that tropifexor improves serum biochemistry parameters, liver damage, and fibrosis in ANIT-induced cholestatic rats.

FIG. 2. shows that tropifexor ameliorates NASH-like symptoms in the SIAM model: NAFLD activity score, hepatic triglycerides, and Sirius Red positive areas and Plasma cholesterol levels were significantly reduced.

FIG. 3. shows that tropifexor reverses fibrosis in a diet-driven insulin resistant model of NASH.

### MODES OF CARRYING OUT THE INVENTION

Liver fibrosis is a key hallmark of advanced liver diseases such as PBC and NASH. In particular, fibrosis drives the prognosis in NAFLD and NASH because it is associated with overall and liver-related morbidity and mortality. Currently, there are no direct anti-fibrotic therapies approved for liver fibrosis; hence, resolution of fibrosis remains a key unmet need in the NASH disease landscape. In this regard, the invertors have found out that tropifexor significantly reduced liver fibrosis as confirmed by a reduction in collagen deposition in a dose-dependent manner in three distinct chronic liver disease models. Furthermore, preclinical studies evaluating higher exposure levels of tropifexor (higher than tropifexor doses disclosed in WO2017145041) demonstrate that greater FXR activation is possible (both in vitro and in vivo) suggesting that increased level of FXR activation result in greater efficacy. In a NASH mouse model higher dosing resulted in lower NAFLD Activity Score and reduced fibrosis. Hepatocellular hypertrophy was only adverse in animal models at exposures (e.g in dogs, Mean AUC0-24 h of 898 and 507 ng*h/mL in males and females respectively) well above the level in NASH patients if treated with triopifexor at a dose of about 140 μg to about 250 μg (e.g. 80 ng*h/mL at 200 μg). For example at the 140 μg and 200 μg doses, approximately 80% and 95% of NASH patients may achieve an AUC>40 ng*h/mL. Therefore, tropifexor at a dose of about 140 μg to about 250 μg is advantageous for the treatment of chronic liver disease, e.g. non-alcoholic fatty liver disease (NAFLD), non-alcoholic steatohepatitis (NASH); furthermore, tropifexor at a dose of about 140 μg to about 250 μg provides a safe and effective treatment when administered to patients.

Tropifexor at a dose of about 140 μg to about 250 μg when administered to a patient with mild to moderate NASH and F2/F3 fibrosis as assessed by histological improvement from baseline shows that about 50% patients with liver fibrosis improvement (at least 1 stage) with no worsening of the NAFLD Activity Score (NAS) or about 30% patients with resolution of NASH (NAS 0 or 1) with no worsening of liver fibrosis.

Tropifexor at a dose of about 140 μg to about 250 μg when administered to a patient with mild to moderate NASH and F2/F3 fibrosis as assessed by histological improvement from baseline shows normalization of liver enzymes in about 50% or more of patients.

Tropifexor at a dose of about 140 μg to about 250 μg when administered to a patient with mild to moderate NASH and F2/F3 fibrosis as assessed by histological improvement from baseline shows reduction of hepatic fat (for example 30% relative reduction; for example 5% absolute reduction).

Tropifexor at a dose of about 140 μg to about 250 μg when administered to a patient with mild to moderate NASH and F2/F3 fibrosis as assessed by histological improvement from baseline shows no significant pruritus outcomes as judged by NASH PRO or 5-D pruritus or visual analogue scale (VAS). The 5-D is a reliable, multidimensional measure of itching that has been validated in patients with chronic pruritus to able to detect changes over time.

The FXR agonists, e.g. tropifexor, may be used in vitro, ex vivo, or incorporated into pharmaceutical compositions and administered to individuals (e.g. human subjects) in vivo to treat, ameliorate, or prevent liver diseases and disorders. A pharmaceutical composition will be formulated to be compatible with its intended route of administration (e.g., oral compositions generally include an inert diluent or an edible carrier). Other nonlimiting examples of routes of administration include parenteral (e.g., intravenous), intradermal, subcutaneous, oral (e.g., inhalation), transdermal (topical), transmucosal, and rectal administration. The pharmaceutical compositions compatible with each intended route are well known in the art. Exemplary pharmaceutical compositions comprising an FXR agonist of formula (I), e.g. tropifexor are described in WO2012/087519.

The frequency of dosing may be twice per day, once per day, or every two days, e.g. once a day. In some embodiments the frequency of dosing is twice per day. The dosing frequency will depend on, inter alia, the phase of the treatment regimen.

In some embodiments, the dosing regimen comprises administration of tropifexor about 140 μg-about 250 μg delivered orally, e.g. about 140 μg-about 200 μg delivered orally. Such doses may be for daily administration (daily doses), or twice daily administration or every two days administration, e.g. for daily administration.

In some embodiments, the dosing regimen comprises administration of tropifexor at a dose in a range of about 140 μg-about 250 μg delivered orally, e.g. about 140 μg-about 200 μg delivered orally. Such doses may be for daily administration (daily doses), or twice daily administration or every two days administration, e.g. for daily administration.

In some embodiments, the dosing regimen comprises administration of tropifexor at a dose of about 140 μg delivered orally, about 150 μg delivered orally, about 160 μg delivered orally, about 170 μg delivered orally, about 180 μg delivered orally, about 190 μg delivered orally, about 200 μg delivered orally, about 210 μg delivered orally, about 220 μg delivered orally, about 230 μg delivered orally, about 240 μg delivered orally or about 250 μg delivered orally. Such doses may be for oral administration.

In some embodiments, the dosing regimen comprises administration of tropifexor at a dose in a range of about 140 μg/day to about 250 μg/day, about 140 μg/day to about 200 μg/day

In some embodiments, the dosing regimen comprises administration of tropifexor at a dose of about 140 μg twice daily, about 150 μg twice daily, about 160 μg twice daily, about 170 μg twice daily, about 180 μg twice daily, about 190 μg twice daily, about 200 μg twice daily, about 210 μg twice daily, about 220 μg twice daily, about 230 μg twice daily, about 240 μg twice daily or about 250 μg twice daily. Such regimens may be delivered orally.

Disclosed herein are methods of treating or preventing a liver disease or disorder as herein above defined, comprising administering a subject in need thereof tropifexor at a dose of about 140 μg/day to about 250 μg/day, about 140 μg/day to about 200 μg/day.

Disclosed herein are methods of treating or preventing a liver disease or disorder as herein above defined, comprising administering a subject in need thereof tropifexor at about 140 μg, about 150 μg, about 160 μg, about 170 μg, about 180 μg, about 190 μg, about 200 μg, about 210 μg, about 220 μg, about 230 μg, about 240 μg or about 250 μg. In some embodiments such a dose is administered daily, e.g. orally. In some embodiments such a dose is administered orally, e.g. daily.

Disclosed herein are FXR agonists of formula (I), a stereoisomer, an enantiomer, a pharmaceutically acceptable salt thereof or an amino acid conjugate thereof, e.g. tropifexor or an amino acid conjugate thereof, for use in treating or preventing a liver disease or disorder as herein above defined, characterized in that tropifexor is to be administered at a dose selected from the group consisting of about 140 μg, about 150 μg, about 160 μg, about 170 μg, about 180 μg, about 190 μg, about 200 μg, about 210 μg, about 220 μg, about 230 μg, about 240 μg or about 250 μg. Such doses may be administered daily, twice daily or every two days, e.g. daily. Such doses may be administered orally.

In some embodiments, is disclosed tropifexor or an amino acid conjugate thereof, e.g. tropifexor, for use in treating or preventing a liver disease or disorder as herein above defined, wherein tropifexor is to be administered at a daily dose selected from the group consisting of about 140 μg, about 200 μg or about 250 μg.

In some embodiments, are disclosed FXR agonists of formula (I), a stereoisomer, an enantiomer, a pharmaceutically acceptable salt thereof or an amino acid conjugate thereof, e.g. tropifexor or an amino acid conjugate thereof, for use in treating or preventing a liver disease or disorder as herein above defined, wherein said FXR agonist is to be administered twice daily at a dose selected from the group consisting of about 140 μg, about 200 μg or about 250 μg. In some embodiments, is disclosed tropifexor or an amino acid conjugate thereof, for use in treating or preventing a liver disease or disorder as herein above defined, wherein tropifexor is to be administered every two days at a dose selected from the group consisting of about 140 μg, about 150 μg, about 160 μg, about 170 μg, about 180 μg, about 190 μg, about 200 μg, about 210 μg, about 220 μg, about 230 μg, about 240 μg or about 250 μg.

In some embodiments, is disclosed FXR agonists of formula (I), a stereoisomer, an enantiomer, a pharmaceutically acceptable salt thereof or an amino acid conjugate thereof, e.g. tropifexor or an amino acid conjugate thereof, for use in treating or preventing a liver disease or disorder as herein above defined, wherein tropifexor is to be administered at a daily dose of about 140 μg or about 200 μg.

In some embodiments, there is provided tropifexor at a daily dose of about 140 μg, of about 200 μg, of about 250 μg.

In some embodiments, there is provided tropifexor at a a daily dose of about 150 μg, about 160 μg, about 170 μg, about 180 μg, about 190 μg, about 200 μg, about 210 μg, about 220 μg, about 230 μg, about 240 μg or about 250 μg for use in treating a chronic liver disease, e.g. non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, e.g. for use in treating non-alcoholic steatohepatitis (NASH) or for use in treating phenotypic NASH.

In some embodiments, there is provided a pharmaceutical unit dosage form composition comprising about 140 μg, about 150 μg, about 160 μg, about 170 μg, about 180 μg, about 190 μg, about 200 μg, about 210 μg, about 220 μg, about 230 μg, about 240 μg or about 250 μg of tropifexor suitable for oral administration up to a maximum total dose of 100 μg per day. Such dosage forms are selected from a liquid, a tablet, a capsule. The dosage forms are for use in treating a chronic liver disease, e.g. non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, e.g. for use in treating non-alcoholic steatohepatitis (NASH).

In some embodiments, there is provided tropifexor at a daily dose of about 10 μg, of about 30 μg, of about 60 μg, or of about 120 μg, for use in treating a chronic liver disease, e.g. non-alcoholic fatty liver disease (NAFLD).

In some embodiments, there is provided tropifexor at a daily dose of about 140 μg, of about 200 μg or of about 250 μg for use in treating non-alcoholic steatohepatitis (NASH).

In some embodiments, there is provided tropifexor administration once daily, morning in a fasting state, at least 30 minutes prior to first beverage, apart from water, and at least 60 minutes prior to the first meal of the day.

In some embodiments, there is provided tropifexor administration once daily, morning in a fasting state, at least 30 minutes prior to first beverage, apart from water, and at least 60 minutes prior to the first meal of the day; e.g. in an amount of about 140 μg, about 150 μg, about 160 μg, about 170 μg, about 180 μg, about 190 μg, about 200 μg, about 210 μg, about 220 μg, about 230 μg, about 240 μg or about 250 μg.

In some embodiments, there is provided tropifexor at a daily dose of about 140 μg, about 150 μg, about 160 μg, about 170 μg, about 180 μg, about 190 μg, about 200 μg, about 210 μg, about 220 μg, about 230 μg, about 240 μg or about 250 μg, for use in treating non-alcoholic steatohepatitis (NASH) once daily, and tropifexor is to be administered morning in a fasting state, at least 30 minutes prior to first beverage, apart from water, and at least 60 minutes prior to the first meal of the day.

In some embodiments, there is provided a use, tropifexor or a method according to any of above embodiments, a pharmaceutical unit dosage form of above embodiments, is administered to humans with impaired hepatic function and wherein tropifexor or an amino acid conjugate thereof, is administered at reduced dose compared to the dose administered to humans without impaired hepatic function. Such impaired hepatic function may be, for example classified by the Child-Pugh system: mild (Child-Pugh A), moderate (Child-Pugh B), severe (Child-Pugh C). The most established approach for categorization of liver impairment is currently the Child-Pugh system. A reduction of the dose in the hepatic impaired subjects is contemplated.

In some embodiments, there is provided tropifexor at a daily dose of about 140 μg, of about 200 μg or of about 250 μg for use in treating non-alcoholic steatohepatitis (NASH) dose and wherein the above dose is reduced to about half in hepatic impaired subjects compared to the dose administered to humans without impaired hepatic function.

Disclosed herein are methods of treating or preventing a liver disease or disorder as herein above defined, in hepatic impaired subjects comprising administering such subject in need thereof tropifexor at a dose of about 70 μg/day to about 120 μg/day, about 70 μg/day to about 100 μg/day.

Kits for the Treatment of Liver Disease or Disorders

Provided herein are kits useful for providing tropifexor for the treatment of a liver disease or disorder as herein above defined. Such kits may comprise tropifexor or an amino acid conjugate therefore a pharmaceutical composition comprising tropifexor. Additionally, such kits may comprise means for administering tropifexor (e.g. solid composition) and instructions for use.

Accordingly, disclosed herein are kits comprising: a) a pharmaceutical composition comprising a therapeutically effective amount of tropifexor or an amino acid conjugate thereof, e.g. tropifexor; b) means for administering tropifexor to a subject a liver disease or disorder as herein above defined; and c) instructions for use, wherein the pharmaceutical composition comprises tropifexor at dose (e.g. daily dose) in a range of about 140 μg to about 250 μg, about 140 μg to about 200 μg.

Are also disclosed kits comprising: a) a pharmaceutical composition comprising a therapeutically effective amount tropifexor or an amino acid conjugate thereof, e.g. tropifexor; b) means for administering tropifexor to a subject having a liver disease or disorder as herein above defined; and c) instructions for use, wherein the pharmaceutical composition comprises a dose of tropifexor selected from the group consisting of about 140 μg, about 150 μg, about 160 μg, about 170 μg, about 180 μg, about 190 μg, about 200 μg, about 210 μg, about 220 μg, about 230 μg, about 240 μg or about 250 μg of the FXR agonist molecule.

In another embodiment, tropifexor or an amino acid conjugate thereof, e.g. tropifexor, is administered enterally; and more particularly, orally.

Unless specified otherwise, a compound for use in the methods of the invention refers to tropifexor or an amino acid conjugate thereof, prodrugs, and inherently formed moieties (e.g., polymorphs, solvates and/or hydrates). Any formula given herein is also intended to represent unlabeled forms as well as isotopically labeled forms of the compounds.

### EXAMPLES

### Example 1

Animal Experiments

Experimental protocols were approved by the local Animal Care and Use Committee and were in compliance with Animal Welfare Act regulations and US regulation (Guide for the Care and Use of Laboratory Animals). Adult male Wistar Han rats (Charles River Laboratories, Inc.) aged 10.6 weeks and weighing ˜300-370 g were randomized and dosed once daily (qd) for 14 days with oral suspensions of tropifexor (0.003, 0.01, 0.03, 0.1, 0.3, 1.0, and 3.0 mg/kg), OCA (0.24, 1.2, 6 and 30 mg/kg), or vehicle using a gavage needle. The tropifexor-treated rats were sacrificed at 1 and 7 h (n=3/time point) and OCA-treated rats were sacrificed at 1, 3, and 7 h (n=3/time point) after the final dose on day 14 for analysis of target gene expression and serum biomarkers. 8-week old, male, Sprague-Dawley (SD) rats (Charles River Laboratories, Inc.) weighing 200-220 g were fed with a modified Picolab rodent diet 5053 containing 0.1% alpha-naphthyl-isothiocyanate (ANIT) to induce severe cholestasis. Beginning with Day 3, tropifexor (LJN452) or OCA was orally gavaged qd for 5 days at doses 0.03, 0.3, and 1 mg/kg (6 rats per group) or 1, 5, and 25 mg/kg (5 rats per group), respectively. Rats were sacrificed 3-5 h after the last dose, blood samples were collected by cardiac puncture, and serum biomarkers of cholestasis, namely alanine transaminase (ALT), aspartate transaminase (AST), alkaline phosphatase (ALP), total bilirubin, total BA, and gamma-glutamyl transpeptidase (GGT) were analyzed.

Further experiments in the STAM model were performed: 2-day old male C57Bl/6J mice were injected with streptozotocin and placed on a high-fat diet (HFD) from weeks 4-12 (HFD-32; CLEA-Japan, Tokyo, Japan). From weeks 9-12, STAM mice received tropifexor (LJN452) 0.03, 0.1, or 0.3 mg/kg; OCA 25 mg/kg; or corresponding vehicles qd orally. Hematoxylin & eosin (H&E)-stained sections were evaluated for nonalcoholic fatty liver disease (NAFLD) activity score (NAS) according to previously defined criteria. Liver total lipid-extracts were isolated, and triglycerides were measured using the Triglyceride E-test (Wako Pure Chemical Industries, Ltd., Japan).

A separate model for diet-induced NASH was developed as described by Trevaskis et al. Male C57B16 mice aged −6 weeks were maintained on a high fat (40% kcal; Primex), high fructose (22% by weight), and high cholesterol (2% by weight) diet (Research Diets Inc., New Brunswick, N.J. cat. no. D09100301) for 26 weeks to induce NASH. Control animals received low-fat diet (10% kcal) with no fructose or cholesterol (Research Diets, cat. no. D09100304). From week 26, animals received tropifexor (LJN452) 0.03, 0.3, or 1.0 mg/kg or OCA 25 mg/kg qd orally for 4 weeks. Expression of collagen, type I, alpha 1 (Col1a1) and tissue inhibitor of metalloproteinase 1 (Timp1) genes was analyzed by real time quantitative PCR.

Histopathology

Liver sections were fixed in 4% paraformaldehyde for 48 h and shipped for histopathological analysis. Liver damage and collagen deposition were assessed by H&E staining and picrosirius red staining, respectively. In the diet-driven NASH model, liver sections were stained with Masson trichrome stain (Sigma-Aldrich, St Louis, Mo., USA) and for ionized calcium binding adaptor molecule 1 (IBA1; Wako cat#019-19741). Quantification of images was done with a positive pixel count algorithm using Aperio software (Aperio, Inc., Vista, Calif.).

Results

Serum biomarkers AST, ALT, total Bile Acids, total bilirubin, and GGT were markedly elevated in vehicle-treated cholestatic (ANIT-treated) animals relative to vehicle-treated non-cholestatic (control) animals (FIG. 1A). Tropifexor treatment at doses as low as 0.3 mg/kg caused a marked reduction in AST, ALT, total BAs, total bilirubin, and GGT levels. Furthermore, at the 1.0 mg/kg dose, levels of most cholestatic markers were not only significantly reduced relative to vehicle-treated ANIT controls, but also normalized to corresponding levels of vehicle-treated non-cholestatic control animals, indicating complete resolution of cholestasis. Liver histology from tropifexor-treated cholestatic rats showed a dose-dependent improvement in necrosis of the bile duct epithelium, bile duct hyperplasia, and presence of inflammatory cell infiltrates in the portal vein regions with respect to livers from vehicle-treated cholestatic rats (FIG. 1B). Additionally, the induction of collagen deposition and liver fibrosis by chronic ANIT treatment (FIG. 1C, top left panel) is highly reduced by tropifexor in a dose-dependent manner (FIG. 1C, right panel). Quantitation of collagen deposition confirmed an increase in fibrosis in vehicle-treated ANIT livers that significantly decreased with LJN452 treatment in a dose-dependent manner (FIG. 1D).

In order to test the effect of OCA on cholestatic biomarkers, a parallel study with varying doses of OCA (1, 5, and 25 mg/kg) was performed using the ANIT model. Mixed effects were observed on serum biochemistry parameters with OCA. Unlike tropifexor, OCA showed significant decrease only in total BA and bilirubin at the 25 mg/kg dose, indicating that tropifexor was more effective than OCA on reduction of cholestatic disease markers.

Treatment with LJN452 showed a significant decrease in NAS at doses 0.1 and 0.3 mg/kg due to reductions in all 3 components of the NAS score (steatosis, lobular inflammation, and hepatocyte ballooning; FIG. 2A). Steatosis improvement was demonstrated by histopathology and reduction in liver triglycerides (FIG. 2A-2B). Importantly, these changes were observed relative to the baseline group indicating a regression in NASH from the baseline (FIG. 2A-2C). High concentration of OCA (25 mg/kg) did not result in a significant decrease in liver triglycerides, but tended toward reduction in NAS (P>0.05), indicating that tropifexor resolves the NASH phenotype more effectively than OCA. The percentage of Sirius Red-positive areas within liver sections was higher in STAM mice relative to normal mice, demonstrating the presence of fibrosis. Tropifexor-treated mice showed a statistically significant dose-dependent reduction of the characteristic pericellular fibrosis observed in STAM. Additionally, fibrosis area of tropifexor-treated mice was decreased in comparison with baseline group (FIG. 2A, 2C), indicating complete regression of the fibrotic phenotype of NASH by tropifexor.

Because many individuals with NASH are obese and diabetic, the effects of tropifexor in an obese, insulin-resistant NASH model have been evaluated. NASH was established in mice by feeding a high trans-fat, high fructose, and high cholesterol diet (AMLN diet) for 26 weeks followed by compound treatment for an additional 4 weeks. Consistent with results from the STAM model, tropifexor resolved liver inflammation, steatosis, and fibrosis in therapeutic mode in this diet-driven model of NASH. Markers of liver damage ALT and AST were elevated in NASH mice compared to control animals that were fed with a low-fat diet (10% fat). Tropifexor-treated NASH mice showed dose-dependent reduction of ALT and AST relative to vehicle-treated controls (FIG. 3A). Importantly, the mid-dose level of tropifexor normalized ALT and AST levels to that of control animals, while the high dose reduced ALT/AST to an even greater extent (FIG. 3A). OCA did not show a statistically significant effect on ALT and AST levels. Further, liver histology analysis showed that the steatosis, ballooning, and inflammation found in vehicle-treated NASH mice were completely reverted by tropifexor at 0.3 and 0.9 mg/kg dose groups (FIG. 3B). Dose-dependent reduction in steatosis by tropifexor was further confirmed by quantification of liver triglycerides (FIG. 3D). By contrast, a high dose of OCA (25 mg/kg) has only a slight effect on reducing steatosis and inflammation.

In addition to steatosis, vehicle-treated NASH groups displayed significant hepatic inflammation as shown by the staining of macrophages and Kupffer cells (IBA+ cells; FIG. 3C). In these groups, macrophages form the characteristic crown-like structures previously described in human and rodent NASH livers. Interestingly, hepatic crown-like structures were completely eliminated in the livers of mid- and high dose tropifexor-treated NASH mice, but not in OCA-treated mice (FIG. 3C). Quantification of IBA positive staining further confirmed the reduction of inflammation by tropifexor that was normalized to the same level of the control diet group with the 0.3 and 0.9 mg/kg tropifexor dose groups (FIG. 3D).

Consistent with previous studies, the AMLN diet induced hepatic fibrosis in this model of NASH (FIGS. 3B, 3D). Trichrome staining showed that tropifexor strongly abrogates collagen deposition in liver (FIGS. 3B, 3D), even to levels lower than that of low-fat diet-treated control mice. Consistent with the histology findings, tropifexor dramatically reduced mRNA levels of fibrogenic markers Col1a1 and TIMP1. Taken together, both STAM and AMLN in vivo studies demonstrate that tropifexor improves NASH via reduction of liver steatosis and the resolution of inflammation and fibrosis.

### Example 2

The safety profile of tropifexor was further evaluated in oral gavage toxicity studies conducted in rats for up to 26 weeks and in dogs for up to 39 weeks.

The data obtained from the NASH mouse model have revealed that dose of 0.3 mg/kg in mice provides exposure of 129 ng*h/mL, which is higher than the predicted exposure of 200 μg daily in NASH patients of approximately 80 ng·hr/ml.

The longer term animal toxicity studies have confirmed that the exposure in NASH patients at a dose of 90 μg maintains a <1-fold safety margin to the rat NOAEL, slightly >1-fold against the previous cap of 70 ng·hr/ml) but of >2-fold safety margin to the dog NOAEL.

### Example 3

It has been shown that the pharmacodynamic marker, FGF19 continues to rise with increasing tropifexor doses up to 3000 μg. An exploratory exposure-response analysis with the biomarker data at week 8 for ALT, AST, FGF19 and GGT in NASH patient treated with tropifexor at doses of 10 μg, 30 μg, 60 μg and 90 μg, have shown that exposures of AUC>40 ng*h/mL provide a maximum biomarker response, thus better treatment effect. At the 140 μg and 200 μg doses, approximately 80% and 95% of NASH patients achieve an AUC>40 ng*h/mL.

Study Protocol

Adult male and female patients with EITHER histologic evidence of NASH on liver biopsy within 2 years prior to randomization and elevated ALT, OR phenotypic diagnosis of NASH based on elevated ALT, Type 2 diabetes mellitus or elevated HbA1c and increased BMI, in both cases accompanied by liver fat >10% on centrally-read MRI.

Diagnosis of NASH: Adequate liver biopsy sample for evaluation by Central Reader to confirm Histologic evidence of NASH based on liver biopsy obtained during the Screening period or within 6 months before randomization with a diagnosis consistent with NASH, fibrosis level F2 or F3, and no diagnosis of alternative chronic liver diseases._AND_ALT≥43 IU/L (males) or 28 IU/L (females).

Patients are assigned at the baseline visit to one of the following 3 treatment arms in a ratio of 1:1:1 in a blinded manner. Placebo capsules will be given to maintain blinding.

Arm 1: Once daily (morning, fasting) treatment with 140 μg tropifexor for 48 weeks

Arm 2: Once daily (morning, fasting) treatment with 200 μg tropifexor for 48 weeks

Arm 3: Once daily (morning, fasting) treatment with matching placebo for 48 weeks Efficacy assessments: The analysis of efficacy variables is be based on descriptive statistics and repeated measures ANCOVA and supported by graphical displays. The efficacy variables are: MRI for hepatic fat fraction, Liver Function Test, Liver histology, Coagulation test, Markers of liver fibrosis, NAFLD Fibrosis score, Fasting lipids, Fasting insulin and glucose, Soluble biomarkers.

### Example 4

Pharmacokinetic models (PBPK model and simCYP model) were established to predict the potential magnitude of PK increase in hepatic impaired subjects in comparison with OCA's liver impairment study results. The PBPK model predicted 1.56-fold increase in AUC and the simCYP model predicted 2.06-fold increase in AUC in severe impaired patients. Therefore, a reduction of the dose in the hepatic impaired subjects is contemplated.

The most established approach for categorization of liver impairment is the Child-Pugh system. This study focuses on subjects with all 3 classes of hepatic impairment.

A single dose of 200 μg of tropifexor is administered to hepatically impaired subjects and their matched healthy counterparts. All 3 classes of hepatically impaired subjects and healthy subjects are enrolled, with Class C subjects enrolled after half of Class A and B subjects are safely dosed. A sufficient number of up to 48 male and female subjects, aged 18 to 70 years, are enrolled in order to ensure at least 6 evaluable subjects per group to complete the study.

TABLE 1
Child-Pugh classification criteria

| | Points scored for each observed finding | | |
|---|---|---|---|
| Finding | 1 | 2 | 3 |
| Encephalopathy¹ | None | 1 or 2 (or suppressed | 3 or 4 (or refractory) |
| | | with medication) | |
| Ascites² | Absent | Slight or subject on 1 | Moderate or severe, |
| | | medication to control | or subject on |
| | | ascites | 2 medications to |
| | | | control ascites |
| Bilirubin (mg/dL) | <2 | 2 to 3 | >3 |
| Albumin (g/dL) | >3.5 | 2.8 to 3.5 | <2.8 |
| INR | <1.7 | 1.7 to 2.2 | >2.2 |

Source: FDA Guidance for Industry 2003, EMA Guideline 2005, FDA/CDR Guidance for Industry 2003, active Guidance 2007

1 Grade 0: normal consciousness, personality, neurological examination, and electroencephalogram.

Grade 1: restless, sleep disturbed, irritable/agitated, tremor, impaired handwriting, 5 cycles/sec waves.

Grade 2: lethargic, time-disoriented, inappropriate, asterixis, ataxia, slow triphasic waves.

Grade 3: somnolent, stuporous, place-disoriented, hyperactive reflexes, rigidity, slower waves.

Grade 4: unarousable coma, no personality/behavior, decerebrate, slow 2 to 3 cycles/sec delta activity.

2 Ascites is graded according to the following criteria: Absent: No ascites detectable by manual investigation. Slight: ascites palpation doubtful. Moderate: ascites detectable by palpation. Severe: necessity of paracentesis, does not respond to medicinal treatment.

### Example 5

FLIGHT-FXR (NCT02855164) is a Phase 2 randomized, double-blind, placebo-controlled trial with an adaptive design of 3 sequential parts to assess safety, tolerability and efficacy in NASH patients. Treatment duration in Parts A & B was 12 weeks. Population included 198 patients (47% male) with liver fat, elevated alanine transaminase (ALT) and NASH on either a historical biopsy or phenotype. Pooled results from treatment arms common to Parts A & B (placebo: 46; TXR 60 μg: 37; TXR 90 μg: 85) were assessed in both pre-specified baseline BMI subgroups for target engagement (fibroblast growth factor-19 [FGF19] and 7-hydroxy-4-cholesten-3-one [C4]), changes from baseline in ALT, gamma-glutamyl transaminase (GGT), liver fat (magnetic resonance imaging-proton density fat fraction [MRI-PDFF]) and safety (See below Table). Statins initiation was not allowed during the trial.

Results in the BMI subgroups are shown in table as geometric mean of percentage (%) changes from baseline to Week 12, except for FGF19 (change 4 hours post dose from pre-dose at Week 6). P-values are not shown because hypothesis testing was not done. Effect of TXR on ALT, GGT and PDFF was more pronounced in subgroup of lower BMI. TXR was well tolerated without safety signals of clinical relevance (including pruritus and lipids).

In both BMI subgroups, TXR results provide evidence of target engagement, anti-inflammatory and anti-steatotic effects with favorable safety and tolerability. Consistent trends of lower responses in sub-group receiving lower dosing by body weight support testing higher TXR doses (140 and 200 μg/d) in the biopsy-based Part C, which may provide improved efficacy without jeopardizing safety.

TABLE 2
Results in the BMI subgroups

| | BMI (kg/ | | | BMI (kg/ | | |
|---|---|---|---|---|---|---|
| | m²) <35 (Non- | | | m²) ≥35(Non- | | |
| | Asian) or <30 (Asian) | | | Asian) or ≥30 (Asian) | | |
| | | TXR | TXR | | TXR | TXR |
| | Placebo | 60 μg | 90 μg | Placebo | 60 μg | 90 μg |
| Stratum | (n = 28) | (n = 21) | (n = 52) | (n = 18) | (n = 16) | (n = 33) |
| FGF19 | 22 | 360 | 586 | 68 | 277 | 447 |
| C4 | 2.8 | −33.2 | −40.4 | 37.3 | −48.9 | −61.8 |
| GGT | −10.8 | −47.0 | −61.3 | −6.8 | −38.4 | −48.7 |
| ALT | −18.6 | −26.0 | −26.8 | −10.6 | −14.8 | −19.5 |
| MRI- | −13.1 | −19.9 | −18.8 | −5.5 | −12.9 | −11.4 |
| PDFF | | | | | | |
| LDL-C | −9.7 | 10.0 | 12.7 | 0.7 | 8.9 | 6.9 |
| HDL-C | −4.8 | −1.9 | −7.7 | −3.9 | −6.1 | −11.9 |
| TG | 1.2 | 1.0 | 5.7 | 0.9 | −6.7 | −2.3 |
| Weight | −0.1 | −1.0 | −1.1 | 0.0 | −1.2 | −1.6 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1-10. (canceled)

11. A method for treating or preventing a condition mediated by Farnesoid X receptor (FXR), comprising administering tropifexor, a pharmaceutically acceptable salt, an amino acid conjugate or an acyl glucuronide conjugate thereof, at a dose of about 140 μg to about 250 μg, to a subject in need thereof; wherein said condition mediated by FXR is a chronic liver disease, drug-induced bile duct injury, gallstones, liver cirrhosis, alcohol-induced cirrhosis, cystic fibrosis, bile duct obstruction, cholelithiasis or liver fibrosis.

12. The method according to claim 11, wherein said condition mediated by FXR is a chronic liver disease.

13. The method according to claim 12, wherein said chronic liver disease is non-alcoholic fatty liver disease (NAFLD).

14. The method according to claim 13, wherein said non-alcoholic fatty liver disease is non-alcoholic steatohepatitis (NASH).

15. The method according to claim 11, comprising administering tropifexor in a free form.

16. The method according to claim 11, comprising administering a glycine or taurine conjugate of tropifexor.

17. The method according to claim 11, comprising administering an acyl glucuronide conjugate of tropifexor.

18. The method according to claim 11, comprising administering about 140 μg, about 150 μg, about 160 μg, about 170 μg, about 180 μg, about 190 μg, about 200 μg about 210 μg, about 220 μg, about 230 μg, about 240 μg or about 250 μg of tropifexor, or a pharmaceutically acceptable salt thereof, up to a maximum total dose of about 500 μg per day.

19. The method according to claim 11, comprising administering about 140 μg to about 200 μg of tropifexor, or a pharmaceutically acceptable salt thereof.

20. The method according to claim 11, comprising administering about 140 μg of tropifexor, or a pharmaceutically acceptable salt thereof.

21. The method according to claim 11, comprising administering about 200 μg of tropifexor, or a pharmaceutically acceptable salt thereof.

22. The method according to claim 11, wherein the dose is a daily dose or twice daily dose.

23. The method according to claim 11, wherein the dose is once every two days.

24. A method for treating non-alcoholic fatty liver disease, comprising administering tropifexor or a pharmaceutically acceptable salt thereof, at a dose of about 140 μg to about 250 μg, to a subject in need thereof.

25. The method of claim 24, wherein said non-alcoholic fatty liver disease is non-alcoholic steatohepatitis.

26. The method according to claim 24, comprising administering about 140 μg to about 200 μg of tropifexor.

27. The method according to claim 24, comprising administering about 140 μg of tropifexor.

28. The method according to claim 24, comprising administering about 200 μg of tropifexor.

29. The method of claim 24, comprising administering once a day about 140 μg of tropifexor.

30. The method of claim 24, comprising administering once a day about 200 μg of tropifexor
